(19) United States
(12) Patent Application Publication
(10) Pub. No.: US 2025/0336501 A1
(43) Pub. Date: Oct. 30, 2025

(54) SYSTEM AND METHOD FOR PATIENT CONDITION MONITORING

(71) Applicant: Reza Farivar-Mohseni, Montreal (CA)

(72) Inventor: Reza Farivar-Mohseni, Montreal (CA)

(21) Appl. No.: 19/189,549

(22) Filed: Apr. 25, 2025

Related U.S. Application Data
(60) Provisional application No. 63/638,989, filed on Apr. 26, 2024.

(51) Int. Cl. G16H 30/40; G06T 7/00; G06V 10/82; G06V 30/18; G06V 40/16; G16H 15/00

(52) U.S. Cl. CPC G16H 30/40; G06T 7/0012; G06V 10/82; G06V 30/18; G06V 40/174; G16H 15/00; G06T 2207/10048

(57) ABSTRACT

A system and method for monitoring a patient are disclosed. The method includes receiving and pre-processing image data captured by a set of cameras positioned to acquire images of either medical instrument or a patient within a clinical environment. The method further includes extracting an instrument identification code from a tamper-proof marking in the image data and validating the code against a pre-registered instrument ID. The method also extracts instrument display data using OCR or a trained machine learning model to identify medical parameters displayed on the instrument. The extracted data is transmitted to a time series database on a room integrator computer or cloud storage for storage and further use. Additionally, the method analyzes facial images of the patient to derive medically relevant data, including facial expressions, skin colour corrected for illumination variations, and facial surface or volume changes based on 3-D depth image data. The time series database stores the analyzed medically relevant data and the instrument display data. A visualization and analysis interface is utilized to assist in clinical decision-making based on the stored data.

### BACKGROUND

The disclosure relates to patient monitoring and more particularly, to instrument data extraction and patient condition monitoring using image processing and machine learning.

In contemporary healthcare environments, continuous and accurate patient monitoring plays a vital role in ensuring timely diagnosis and appropriate treatment interventions. Existing patient monitoring systems are primarily designed to capture physiological parameters such as heart rate, oxygen saturation, and body temperature. However, these systems typically focus exclusively on the patient and fail to account for the presence and influence of healthcare personnel, visitors, and other non-patient individuals within the clinical environment.

Furthermore, conventional patient monitoring solutions do not provide mechanisms for seamless, and privacy-compliant video-based behavior capture of non-patient individuals, nor do they offer real-time analytical tools for assessing staff performance or inferring patient behavior based on environmental interactions.

In addition, modern hospital environments rely on a wide range of specialized, disconnected monitoring devices, resulting in fragmented data silos and an absence of unified real-time integration between wired physiological data and visual behavior data. This fragmentation significantly limits the ability to generate automated, clinically actionable insights.

Even when such data is collected, existing visualization and analysis tools often remain unintuitive, disjointed, and lacking in integrated statistical analysis, state inference, and context-aware presentation, which can hinder clinical decision-making.

Therefore, there is a need to address these deficiencies and provide an integrated system and method that can comprehensively monitor the patient and capture and analyze non-patient behavior. Further, there is a need for a system and method to provide an intelligent, and interactive patient console to facilitate real-time visualization, analysis, and inference of patient state. Furthermore, there is a need for a system and method to facilitate these components to operate in synchrony to enhance clinical workflows.

### SUMMARY

According to an embodiment of the disclosure, a computer-implemented method for monitoring a patient is described. The computer-implemented method includes receiving and pre-processing, by a computer, image data captured by a set of cameras. In one aspect, the image data is captured via a wired connection, a network connection, or any combination thereof, in addition to being acquired from the set of cameras. The cameras are configured to capture images of at least one instrument and a patient within a clinical environment. In an aspect, the patient data or vitals may be captured through a wired connection. In scenarios where instruments such as infusion pumps or similar devices are absent, the system and method present invention remain functional by integrating visual data of the patient such as facial and body features with vital signs data. These vital signs may be obtained through a direct (wired) connection, a network connection, or a combination thereof. This multimodal data fusion ensures continuous patient monitoring and system reliability, even in the absence of equipment-based data sources. The computer-implemented method further includes extracting, by the computer, an instrument identification code from a tamper-proof marking present in the pre-processed image data and validating the instrument identification code against a pre-registered instrument ID. In an aspect, the instrument identification code may be extracted through the embedded computer or microcontroller present in the camera module of the set of cameras. The computer-implemented method further includes extracting, by the computer, instrument display data using one or more of an optical character recognition (OCR) algorithm, and a trained machine learning model to identify a set of medical parameters displayed on the instrument. The computer-implemented method further includes transmitting, by the computer, the extracted instrument display data to a time series database on a room integrator computer or in a cloud database. The computer-implemented method further includes analyzing, by the computer, a set of facial images of the patient to derive medically relevant data. The computer-implemented method further includes storing the analyzed medically relevant data and the instrument display data in the time series database and utilizing a visualization and analysis interface to assist in clinical decision-making based on the stored data.

According to one or more embodiments of the disclosure, a computer system for monitoring a patient is described. The computer system includes a processor set, a computer-readable storage media, and program instructions that are stored on the one or more computer-readable storage media. The program instructions are executable by the processor set to cause the processor set to receive and pre-process image data captured by a camera. The camera is configured to capture images of at least one instrument and a patient within a clinical environment. The program instructions further cause the processor set to extract an instrument identification code from a tamper-proof marking present in the pre-processed image data and validate the instrument identification code against a pre-registered instrument ID. The program instructions further cause the processor set to extract instrument display data using one or more of an optical character recognition (OCR) algorithm, and a trained machine learning model to identify a set of medical parameters displayed on the instrument. The program instructions further cause the processor set to transmit the extracted instrument display data to a time series database on a room integrator computer or a cloud storage. The program instructions further cause the processor set to analyze a set of facial images of the patient to derive medically relevant data or useful data. The program instructions further cause the processor set to store the analyzed medically relevant data and the instrument to display data in the time series database and utilize a visualization and analysis interface to assist in clinical decision-making based on the stored data.

According to one or more embodiments of the disclosure, a computer program product for monitoring a patient is described. The computer program product includes a computer-readable storage media or cloud storage having program instructions stored on the computer-readable storage media to perform operations. The operations include receive and pre-process image data captured by a camera. The camera is configured to capture images of at least one instrument and a patient within a clinical environment. The operations further include extract an instrument identification code from a tamper-proof marking present in the pre-processed image data and validate the instrument identification code against a pre-registered instrument ID. The operations further include extract instrument display data using one or more of an optical character recognition (OCR) algorithm, and a trained machine learning model to identify a set of medical parameters displayed on the instrument. The operations further include transmit the extracted instrument display data to a time series database on a room integrator computer or cloud storage. The operations further include analyze a set of facial images of the patient to derive medically relevant data.

Additional technical features and benefits are realized through the process of the disclosure. Embodiments and aspects of the disclosure are described in detail herein and are considered a part of the claimed subject matter. For a better understanding, refer to the detailed description and the drawings.

### BRIEF DESCRIPTION OF THE DRAWINGS

The following description will provide details of preferred embodiments with reference to the following figures wherein:

FIG. 1 is a diagram that illustrates a computing environment for monitoring a patient, in accordance with an embodiment of the disclosure;

FIG. 2 is a diagram that illustrates a network environment for monitoring the patient, in accordance with an embodiment of the disclosure;

FIG. 3A is a diagram that depicts a user interface 300A of a main console pertaining to patient registration and key event overview, in accordance with an embodiment of the disclosure;

FIG. 3B is a diagram that depicts a user interface 300B of clinical parameters and interventions, in accordance with an embodiment of the disclosure;

FIG. 3C is a diagram that depicts a user interface 300C of an example of an analysis window, looking at the correlation between parameters, in accordance with an embodiment of the disclosure;

FIG. 3D is a diagram that depicts a user interface 300D of patient state parameters, in accordance with an embodiment of the disclosure;

FIG. 4 illustrates a flowchart that illustrates a method for monitoring a patient, in accordance with an embodiment of the disclosure; and

FIG. 5 illustrates a flowchart that illustrates an exemplary method for analyzing the facial images of the patient, in accordance with an embodiment of the disclosure.

### DETAILED DESCRIPTION

The proposed system provides an integrated patient monitoring system designed to enhance clinical safety, data integrity, and real-time situational awareness within healthcare environments. In some embodiments, the system includes a room monitoring subsystem, a centralized room integrator computer, and an intelligent patient console. The room monitoring subsystem utilizes one or more cameras, synchronized with a patient body capture system, to authenticate the identities of patients and authorized staff through biometric, RFID, or identification-based methods. The system applies exclusion techniques such as masking, pixelation, and AI-generated substitutions to obscure individuals who are neither patients nor registered personnel while preserving relevant contextual behavior cues. The room integrator computer serves as a secure data aggregation hub, receiving both visual data and wired medical device data from authenticated sources. Unauthorized device modifications trigger alerts, ensuring system integrity. Time-stamped data is stored in a high-performance time series database optimized via a data collection middleware. The patient console provides clinicians with an interactive interface for real-time visualization, analysis, and inference of patient states. The console supports an integrated display of physiological data, advanced statistical and machine learning-based state prediction, manual data entry, EMR synchronization, and customizable analysis pipelines, empowering healthcare professionals with early-warning alerts and enhanced decision support.

One advantage of the proposed system is that it captures, and identifies the authorized staff while masking or substituting non-authorized individuals, thus respecting privacy while maintaining context. The room integrator computer provides a unified data pipeline for direct wired connections to multiple medical devices, ensuring secure data collection, device authentication, and integrity verification.

Accordingly, one advantage of the present invention is that it provides a combination of time series databases with structured pipelines for efficient write and retrieval operations to ensure scalable and query-efficient storage for both visual and device-based patient data. The present invention further streams the data to a cloud or remote storage.

Accordingly, one advantage of the present invention is that it provides a single point of access for visualization, analysis, inference, and reporting of patient physiological data and behavioral states, incorporating statistical tools and advanced machine learning models for mental state identification and clinical decision support.

Accordingly, one advantage of the present invention is that it enables the creation, implementation, loading, execution, and sharing of custom or third-party data analysis pipelines, fostering a collaborative and reproducible clinical data analysis environment.

Accordingly, one advantage of the present invention is that it uses statistical models and AI to infer patient mental states, arousal, and discomfort to provide clinical staff with actionable insights in real-time.

Various aspects of the disclosure are described by narrative text, flowcharts, block diagrams of computer systems, and/or block diagrams of the machine logic included in computer program product (CPP) embodiments. With respect to any flowcharts, depending upon the technology involved, the operations can be performed in a different order than what is shown in a given flowchart. For example, again depending upon the technology involved, two operations shown in successive flowchart blocks may be performed in reverse order, as a single integrated operation, concurrently, or in a manner at least partially overlapping in time.

A computer program product embodiment (“CPP embodiment” or “CPP”) is a term used in the disclosure to describe any set of one, or more, storage media (also called “mediums”) collectively included in a set of one, or more, storage devices that collectively include machine readable code corresponding to instructions and/or data for performing computer operations specified in a given CPP claim. A “storage device” is any tangible device that can retain and store instructions for use by a computer processor. Without limitation, the computer-readable storage medium may be an electronic storage medium, a magnetic storage medium, an optical storage medium, an electromagnetic storage medium, a semiconductor storage medium, a mechanical storage medium, or any suitable combination of the foregoing. Some known types of storage devices that include these mediums include diskette, hard disk, random access memory (RAM), read-only memory (ROM), erasable programmable read-only memory (EPROM or Flash memory), static random-access memory (SRAM), compact disc read-only memory (CD-ROM), digital versatile disk (DVD), memory stick, floppy disk, mechanically encoded device (such as punch cards or pits/lands formed in a major surface of a disc) or any suitable combination of the foregoing. In an embodiment, the storage could be local to the room, local to the hospital or hospital system, or hosted on a third-party cloud platform. A computer-readable storage medium, as that term is used in the disclosure, is not to be construed as storage in the form of transitory signals per se, such as radio waves or other freely propagating electromagnetic waves, electromagnetic waves propagating through a waveguide, light pulses passing through a fiber optic cable, electrical signals communicated through a wire, and/or other transmission media. As will be understood by those of skill in the art, data is typically moved at some occasional points in time during normal operations of a storage device, such as during access, de-fragmentation, or garbage collection, but this does not render the storage device as transitory because the data is not transitory while it is stored.

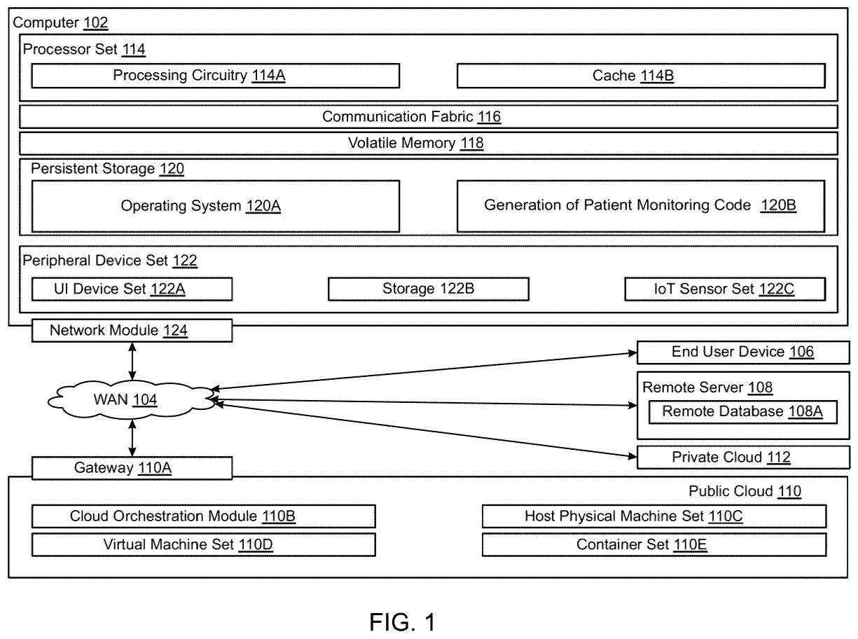

FIG. 1 is a diagram that illustrates a computing environment 100 for monitoring a patient, in accordance with an embodiment of the disclosure. With reference to FIG. 1, there is shown a computing environment 100 that contains an example of an environment for the execution of at least some of the computer code involved in performing the disclosed methods, such as a patient monitoring code 120B. In addition to the patient monitoring code 120B, computing environment 100 includes, for example, a computer 102, a wide area network (WAN) 104, an end-user device (EUD) 106, a remote server 108, a public cloud 110, and a private cloud 112. In this embodiment of the disclosure, the computer 102 includes a processor set 114 (including a processing circuitry 114A and a cache 114B), a communication fabric 116, a volatile memory 118, a persistent storage 120 (including an operating system 120A and the generation of patient monitoring code 120B, as identified above), a peripheral device set 122 (including a user interface (UI) device set 122A, a storage 122B, and an Internet of Things (IoT) sensor set 122C), and a network module 124. The remote server 108 includes a remote database 108A. The public cloud 110 includes a gateway 110A, a cloud orchestration module 110B, a host physical machine set 110C, a virtual machine set 110D, and a container set 110E.

Computer 102 may take the form of a desktop computer, a laptop computer, a single-board computer, an embedded system, a microcontroller, a tablet computer, a smartphone, a smartwatch or other wearable computer, a mainframe computer, a quantum computer, a camera, or any other form of a computer or a mobile device now known or to be developed in the future that is capable of running a program, accessing a network or querying a database, such as a remote database 108A. As is well understood in the art of computer technology, and depending upon the technology, the performance of a computer-implemented method may be distributed among multiple computers and/or between multiple locations. On the other hand, in this presentation of the computing environment 100, detailed discussion is focused on a single computer, specifically the computer 102, to keep the presentation as simple as possible. The computer 102 may be located in a cloud, even though it is not shown in a cloud in FIG. 1. On the other hand, computer 102 is not required to be in a cloud except to any extent as may be affirmatively indicated.

The processor set 114 includes one, or more, computer processors or camera image processors of any type now known or to be developed in the future. The processing circuitry 114A may be distributed over multiple packages, for example, multiple, coordinated integrated circuit chips. The processing circuitry 114A may implement multiple processor threads and/or multiple processor cores. The cache 114B may be memory that is located in the processor chip package(s) and is typically used for data or code that should be available for rapid access by the threads or cores running on the processor set 114. Cache memories are typically organized into multiple levels depending upon relative proximity to the processing circuitry 114A. Alternatively, some, or all, of the cache 114B for the processor set 114 may be located “off-chip.” In some computing environments, the processor set 114 may be designed for working with GPU RAM, qubits, and performing quantum computing.

Computer readable program instructions are typically loaded onto the computer 102 to cause a series of operations to be performed by the processor set 114 of the computers 102 and thereby effect a computer-implemented method, such that the instructions thus executed will instantiate the methods specified in flowcharts and/or narrative descriptions of computer-implemented methods included in this document (collectively referred to as “the disclosed methods”). These computer-readable program instructions are stored in various types of computer-readable storage media, such as the cache 114B and the other storage media discussed below. The program instructions, and associated data, are accessed by the processor set 114 to control and direct the performance of the disclosed methods. In computing environment 100, at least some of the instructions for performing the disclosed methods may be stored in the dynamic modification of the patient monitoring code 120B in persistent storage 120.

The communication fabric 116 is the signal conduction path that allows the various components of computer 102 to communicate with each other. Typically, this fabric is made of switches and electrically conductive paths, such as the switches and electrically conductive paths that make up buses, bridges, physical input/output ports, and the like. Other types of signal communication paths may be used, such as fiber optic communication paths and/or wireless communication paths.

The volatile memory 118 is any type of volatile memory now known or to be developed in the future. Examples include dynamic type random access memory (RAM) or static type RAM. Typically, the volatile memory 118 is characterized by a random access, but this is not required unless affirmatively indicated. In computer 102, the volatile memory 118 is located in a single package and is internal to computer 102, but alternatively or additionally, the volatile memory 118 may be distributed over multiple packages and/or located externally with respect to computer 102.

The persistent storage 120 is any form of non-volatile storage for computers that is now known or to be developed in the future. The non-volatility of this storage means that the stored data is maintained regardless of whether power is being supplied to computer 102 and/or directly to the persistent storage 120. The persistent storage 120 may be a read-only memory (ROM), but typically at least a portion of the persistent storage 120 allows the writing of data, deletion of data, and re-writing of data. Some familiar forms of the persistent storage 120 include magnetic disks and solid-state storage devices. The operating system 120A may take several forms, such as various known proprietary operating systems or open-source Portable Operating System Interface-type operating systems that employ a kernel. The code included in the generation of patient monitoring code 120B typically includes at least some of the computer code involved in performing the disclosed methods.

The peripheral device set 122 includes the set of peripheral devices of computer 102. Data communication connections between the peripheral devices and the other components of computer 102 may be implemented in various ways, such as Bluetooth connections, Near-Field Communication (NFC) connections, connections made by cables (such as universal serial bus (USB) type cables), insertion-type connections (for example, secure digital (SD) card), connections made through local area communication networks and even connections made through wide area networks such as the internet. In various embodiments of the disclosure, the UI device set 122A may include components such as a display screen, speaker, microphone, wearable devices (such as goggles and smartwatches), keyboard, mouse, printer, touchpad, game controllers, and haptic devices. Storage 122B is external storage, such as an external hard drive, or insertable storage, such as an SD card. The storage 122B may be persistent and/or volatile. In some embodiments of the disclosure, storage 122B may take the form of a quantum computing storage device for storing data in the form of qubits. In embodiments of the disclosure where computer 102 is required to have a large amount of storage (for example, where computer 102 locally stores and manages a large database) then this storage may be provided by peripheral storage devices designed for storing very large amounts of data, such as a storage area network (SAN) that is shared by multiple, geographically distributed computers. The IoT sensor set 122C is made up of sensors that can be used in Internet of Things applications. For example, one sensor may be a thermometer and another sensor may be a motion detector.

The network module 124 is the collection of computer software, hardware, and firmware that allows computer 102 to communicate with other computers through WAN 104. The network module 124 may include hardware, such as modems or Wi-Fi signal transceivers, software for packetizing and/or de-packetizing data for communication network transmission, and/or web browser software for communicating data over the internet. In some embodiments of the disclosure, network control functions, and network forwarding functions of the network module 124 are performed on the same physical hardware device. In various embodiments of the disclosure (for example, embodiments that utilize software-defined networking (SDN)), the control functions and the forwarding functions of the network module 124 are performed on physically separate devices, such that the control functions manage several different network hardware devices. Computer-readable program instructions for performing the disclosed methods can typically be downloaded to computer 102 from an external computer or external storage device through a network adapter card or network interface included in the network module 124.

The WAN 104 is any wide area network (for example, the internet) capable of communicating computer data over non-local distances by any technology for communicating computer data, now known or to be developed in the future. In some embodiments of the disclosure, the WAN 104 may be replaced and/or supplemented by local area networks (LANs) designed to communicate data between devices located in a local area, such as a Wi-Fi network. The WAN 104 and/or LANs typically include computer hardware such as copper transmission cables, optical transmission fibers, wireless transmission, routers, firewalls, switches, gateway computers, and edge servers.

The EUD 106 is any computer system that is used and controlled by an end user (for example, a customer of an enterprise that operates computer 102) and may take any of the forms discussed above in connection with computer 102. The EUD 106 typically receives helpful and useful data from the operations of computer 102. For example, in a hypothetical case where computer 102 is designed to provide a recommendation to an end user, this recommendation would typically be communicated from the network module 124 of computer 102 through WAN 104 to EUD 106. In this way, the EUD 106 can display, or otherwise present recommendations to an end user. In some embodiments of the disclosure, EUD 106 may be a client device, such as a thin client, heavy client, mainframe computer, desktop computer, and so on.

The remote server 108 is any computer system that serves at least some data and/or functionality to the computer 102. The remote server 108 may be controlled and used by the same entity that operates the computer 102. The remote server 108 represents the machine(s) that collect and store helpful and useful data for use by other computers, such as the computer 102. For example, in a hypothetical case where the computer 102 is designed and programmed to provide a recommendation based on historical data, then this historical data may be provided to the computer 102 from the remote database 108A of the remote server 108.

The public cloud 110 is any computer system available for use by multiple entities that provides on-demand availability of computer system resources and/or other computer capabilities, especially data storage (cloud storage) and computing power, without direct active management by the user. Cloud computing typically leverages the sharing of resources to achieve coherence and economies of scale. The direct and active management of the computing resources of the public cloud 110 is performed by the computer hardware and/or software of the cloud orchestration module 110B. The computing resources provided by the public cloud 110 are typically implemented by virtual computing environments that run on various computers making up the computers of the host physical machine set 110C, which is the universe of physical computers in and/or available to the public cloud 110. Virtual computing environments (VCEs) typically take the form of virtual machines from the virtual machine set 110D and/or containers from the container set 110E. It is understood that these VCEs may be stored as images and may be transferred among and between the various physical machine hosts, either as images or after the instantiation of the VCE. The cloud orchestration module 110B manages the transfer and storage of images, deploys new instantiations of VCEs, and manages active instantiations of VCE deployments. Gateway 110A is the collection of computer software, hardware, and firmware that allows public cloud 110 to communicate through WAN 104.

Some further explanation of virtualized computing environments (VCEs) will now be provided. VCEs can be stored as “images”. A new active instance of the VCE can be instantiated from the image. Two familiar types of VCEs are virtual machines and containers. A container is a VCE that uses operating-system-level virtualization. This refers to an operating system feature in which the kernel allows the existence of multiple isolated user-space instances, called containers. These isolated user-space instances typically behave as real computers from the point of view of programs running in them. A computer program running on an ordinary operating system can utilize all resources of that computer, such as connected devices, files and folders, network shares, CPU power, and quantifiable hardware capabilities. However, programs running inside a container can only use the contents of the container and devices assigned to the container, a feature which is known as containerization.

The private cloud 112 is similar to public cloud 110, except that the computing resources are only available for use by a single enterprise. While the private cloud 112 is depicted as being in communication with the WAN 104, in various embodiments of the disclosure, a private cloud may be disconnected from the internet entirely and only accessible through a local/private network. A hybrid cloud is a composition of multiple clouds of different types (for example, private, community, or public cloud types), often respectively implemented by different vendors. Each of the multiple clouds remains a separate and discrete entity, but the larger hybrid cloud architecture is bound together by standardized or proprietary technology that enables orchestration, management, and/or data/application portability between the multiple constituent clouds. In this embodiment of the disclosure, the public cloud 110 and the private cloud 112 are both part of a larger hybrid cloud.

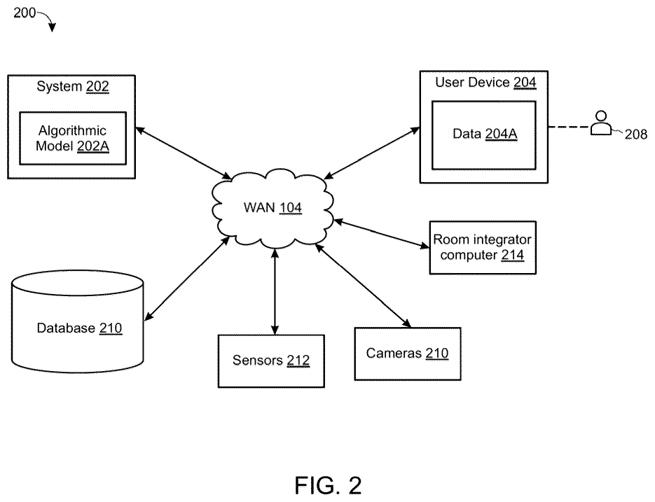

FIG. 2 is a diagram that illustrates a network environment 200 for monitoring the patient, in accordance with an embodiment of the disclosure. FIG. 2 is explained in conjunction with elements from FIG. 1. With reference to FIG. 2, there is shown a diagram of a network environment 200. The network environment 200 includes a computer system (hereinafter referred to as system 202), and a user device 204. The system 202 further includes an algorithmic model 202A. There is further shown a database 210. The network environment 200 further includes entity 208 associated with the user device 204. The network environment 200 further includes the WAN 104 of FIG. 1. In an embodiment of the disclosure, the user device 204 may be an exemplary embodiment of the EUD 106. Similarly, computer system 202 may be an embodiment of computer 102 in FIG. 1. Examples of entity 208 include but are not limited to medical administrators or medical practitioners. In an embodiment, the End User Device (EUD) 106 is configured as a computing system utilized by a human operator or clinical staff member, such as a nurse, doctor, or technician, to access, view, and potentially control or interface with the various components of the patient monitoring ecosystem. The EUD 106 may be implemented in any form factor compatible with the computing architecture described in connection with computer 102 or system 202. The EUD 106 is communicatively coupled with the cameras 210 and sensors 212 deployed in the clinical environment for comprehensive patient and device monitoring. One set of cameras is directed toward care-related devices, such as infusion pumps, perfusion pumps, or dialysis machines. These cameras are configured to capture the data displayed on these devices. The captured visual data is processed using computer vision techniques, such as optical character recognition (OCR) or machine learning algorithms, to extract information about treatment parameters, intervention settings, or device alerts. The processed data is transmitted to the EU D 106, allowing clinical staff to monitor and analyze interventions in real-time. Another group of cameras is dedicated to capturing the facial data of the patient. These include compound camera assemblies such as RGB cameras, infrared cameras, and depth-sensing modules, where the depth data is captured through a stereo arrangement of infrared cameras. These cameras are used to monitor facial expressions, skin tone, and other indicators of patient state, such as discomfort or distress. The EUD 106 receives and displays this multimodal data, which may also be used in conjunction with patient identification or condition monitoring algorithms. Additionally, the system 202 includes cameras directed toward the patient's body to capture body pose and movement. These cameras generate data that can be processed using pose estimation techniques to infer patient posture, and movement trends, or detect sudden changes in position. The EUD 106 allows users to access and review this data, providing insights into patient activity levels, restfulness, or potential risks such as falls or improper positioning. To monitor fluid levels, cameras 210 are also positioned to observe IV bags, fluid containers, or drainage systems. The visual data is analyzed to detect fluid levels, volume changes, or potential leaks. The results are transmitted to the EUD 106, which serves as a visualization and alert platform for clinical staff, helping them track and manage fluid administration effectively. In addition to camera-based monitoring, the system supports direct digital interfaces with clinical devices such as patient vital sign monitors and ventilators. These direct connections allow structured data acquisition with high accuracy and real-time responsiveness. Data from these sources is collected and displayed on the EUD 106, enabling clinical decision-making based on up-to-date physiological information. Finally, the system 202 may also include integration with sensors 212, such as those measuring room temperature, humidity, or noise levels. These sensors provide contextual information about the patient's surroundings, which may affect patient comfort or clinical outcomes. The EUD 106 aggregates and presents this data alongside patient-specific information to offer a holistic view of the care environment.

The system 202 may include suitable logic, circuitry, interfaces, and/or code that may be configured for the monitoring of the patients. System 202 is configured to record and analyze the behavior of non-patient individuals in a medical environment. The system 202 is configured for visualization and analysis of patient data. The system 202 is further configured for the acquisition and storage of patient data.

In an embodiment, the examples of the computer system 202 may include, but are not limited to, a server, a computing device, a virtual computing device, a mainframe machine, a computer workstation, a smartphone, a camera, a cellular phone, a mobile phone, a gaming device, or a consumer electronic (CE) device.

The user device 204 may include suitable logic, circuitry, interfaces, and/or code that may enable users to interact with the system 202. Examples of the user device 204 may include, but are not limited to, a computing device, a mainframe machine, a server, a computer work-station, a smartphone, a camera, a cellular phone, a mobile phone, a gaming device, a consumer electronic (CE) device, a head-mounted device, a Virtual Reality (VR) Headset, an Augmented Reality (AR) Device, a Mixed Reality (MR) Device, a Projection-based System, and/or any other device with computer vision display capabilities.

The display screen may include suitable logic, circuitry, and interfaces that may be configured to render an output generated by the system 202. In some embodiments of the disclosure, the display screen may be an external display device associated with the first user device 204 and the second user device 206. The display screen may be a touch screen which may enable entity 208A to interact via the display screen. The touch screen may be at least one of a resistive touch screen, a capacitive touch screen, or a thermal touch screen. In accordance with an embodiment of the disclosure, the display screen may refer to a display screen of a head-mounted device (HMD), a smart-glass device, a see-through display, a projection-based display, an electrochromic display, or a transparent display. In some embodiments of the disclosure, the display screen may be realized through several known technologies such as, but are not limited to, at least one of a liquid Crystal Display (LCD) display, a Light Emitting Diode (LED) display, a plasma display, or an Organic LED (OLED) display technology.

In an embodiment, the algorithmic model 202A may correspond to a computer-based system or software that exhibits characteristics commonly associated with human intelligence. The algorithmic model 202A may be designed to perform tasks that typically require human intelligence, such as problem-solving, learning, reasoning, perception, understanding natural language, and decision-making. AI systems can range from simple rule-based programs to sophisticated, self-learning systems.

The algorithmic model 202A may be a sophisticated piece of software that leverages natural language processing (NLP) and machine learning processes to understand, generate, and manipulate human language. For example, the algorithmic model 202A may correspond to a large language model (LLM) model that is specifically designed for tasks related to language understanding and generation on a large scale. Certain characteristics of the LLM model may include, but are not limited to, natural language understanding, text generation, semantic understanding, transfer learning, multimodal capabilities, continuous learning, and user interaction.

In an embodiment, the database 210 corresponds to an organized collection of data that may be stored and accessed electronically from a computer system (such as the system 202). The database 210 is configured to manage, store, retrieve, and update data efficiently. In an exemplary implementation, the structure of the database 210 typically involves tables, records, and fields that can be managed through various database management systems (DBMS). Examples of database 210 include but are not limited to, a relational database, a Non-Structured Query Language (SQL) database, a hierarchical database, a network database, a transactional database, a data warehouse, and a distributed database. In an embodiment, the database 210 is configured to store the data 204A associated with the user device 204 which may include the operating system data, software application data, and software application version data. Further, database 210 stores captured images that may be used to train the algorithmic model 202A.

In operation, the system 202 is configured to receive and pre-process image data captured by a set of cameras 210. The cameras 210 are configured to capture images of at least one instrument and a patient within a clinical environment. In one embodiment, a dedicated imaging system is deployed wherein a single camera is configured to monitor a specific set of medical instruments. For example, one camera may be capable of reading data from approximately three infusion pumps. Additional cameras may be integrated into the system to accommodate and monitor a greater number of instruments as needed. In another embodiment, separate cameras are employed for fluid analysis, which may include spectral imaging cameras configured to perform spectral analysis of intravenous fluids, blood, or other administered substances to assess composition, clarity, or contamination. Further, in one embodiment, one or more cameras (such as infrared cameras, time-of-flight sensors, or structured-light 3D cameras) are positioned to capture the facial features of the patient. These cameras may be used to monitor facial expressions, skin tone changes, or signs of discomfort or distress. Additionally, at least one camera is oriented to capture the patient's body, enabling continuous observation of posture, movement, and overall physical state. This body-monitoring camera may be used in conjunction with posture analysis algorithms or movement-tracking systems to infer changes in the patient's condition.

System 202 is further configured to extract an instrument identification code from a tamper-proof marking present in the pre-processed image data and validate the instrument identification code against a pre-registered instrument ID. By way of an example and not limitation, the pre-processing steps include but are not limited to grayscale conversion, noise reduction, binarization, de-skewing, dilation, erosion, shadow removal, lighting adjustment, text region detection, rescaling, and contrast adjustment. The system 202 is further configured to extract instrument display data using one or more of an optical character recognition (OCR) algorithm, and a trained machine learning model to identify a set of medical parameters displayed on the instrument. The system 202 is further configured to transmit the extracted instrument display data to a time series database on a room integrator computer 214. The system 202 is further configured to analyze a set of facial images of the patient to derive medically relevant data. In an embodiment, the extracted instrument display data is transmitted from the processor set to the room integrator computer 214 via one or more of: an encrypted cabled connection, and an encrypted wireless connection. In an embodiment, the infrared images are captured by a compound camera system comprising a set of infrared cameras, a set of 3D cameras, and a set of multispectral imaging systems. In an embodiment, the calibration sticker comprises an array of colour samples with predetermined and fixed reflectance properties. The system 202 is further configured to store the analyzed medically relevant data and the instrument displays data in the time series database and utilizes a visualization and analysis interface to assist in clinical decision-making based on the stored data.

By way of an example and not limitation, the OCR algorithm is Tesseract, EasyOCR, or a re-trained OCR model adapted to the specific instrument type. By way of an example and not limitation, the machine learning model for instrument data extraction is a neural network trained to recognize alphanumeric strings, symbols, or icons displayed on medical instruments. In an embodiment, the facial expression analysis algorithm is optimized for execution on a GPU using CUDA or a similar parallel computing framework. In an embodiment, the colour calibration enables correction for ambient light spectrum variability and allows accurate measurement of patient skin colour for clinical assessment. In an embodiment, the 3-D depth data is obtained from a stereo camera pair or a structured light-based depth camera. The 3-D depth data is further used for automated detection and characterization of facial expressions. By way of an example and not limitation, the time series database is selected from the group consisting of TimescaleDB, GridDB, InfluxDB, OpenTSDB, CrateDB, or a custom-built database.

According to one embodiment, the present invention may provide a body capture camera system for patient monitoring within clinical environments. The camera system may include either a two-dimensional (2-D) camera or a three-dimensional (3-D) depth-sensing camera, such as stereo vision cameras, time-of-flight (ToF) cameras, structured light cameras, or LiDAR-based cameras. In some embodiments, the system may incorporate one or more of these depth sensors or a combination thereof, including multiple 2-D cameras to enhance data fidelity. The camera is mounted on one end of a swing arm constructed from a rigid, sterilizable material, with the opposite end of the swing arm attached to a ball joint or another mechanically adjustable joint that provides both stability and flexible positioning. The joint may further be connected to an auxiliary arm secured to either a patient bed or a nearby instrument mount, enabling precise adjustment of the camera orientation. In some embodiments, a directional light source (such as a laser, collimated beam, or LED) assists clinicians in positioning the camera optimally toward the patient's body. Recognizing the critical need for unobstructed access to the patient, particularly in intensive care settings, the system is designed for rapid detachment and repositioning of the camera assembly. The swing arm and connector work together to allow safe and quick displacement of the camera, minimizing interference during emergency care while reducing risks associated with cable entanglement and tripping hazards. The camera system further includes a data connector that supports various industry-standard transmission protocols, including USB (ranging from USB 2.0 to USB 4.0), FireWire, ThunderBolt, CameraLink, Ethernet, GigE, or custom-designed interfaces based on Serial Peripheral Interface (SPI) or comparable communication standards. The connector employs a magnetic “MagSafe”-type mechanical interface, wherein the male and female portions of the connector are held together magnetically, allowing for quick and safe disconnection without damaging the camera or harming the patient during repositioning. The camera housing is used for both functional utility and patient comfort. The camera further includes LED status indicators located on the rear side of the housing, facing away from the patient to reduce visual disturbance while providing clinical staff with clear visual feedback. The LED indicators are configured to display various operational statuses, including camera power status, authentication of the object in view, successful data capture, and error states such as authentication or capture failure. The camera system's electronic interface supports both wired and wireless data transmission. Wired connections may leverage USB, FireWire, ThunderBolt, GigE, CameraLink, or similar protocols, while wireless transmission may employ Wifi, Bluetooth, or comparable communication standards. In all wired embodiments, the connector utilizes the aforementioned magnetic detachment system, enabling fast, safe removal to allow clinicians immediate physical access to the patient while minimizing the presence of loose cables and reducing fall hazards around the patient's bedside.

According to one embodiment, the present invention may provide a safety-enhancing component to ensure rapid and unhindered access to a patient during urgent medical situations. In such scenarios, the camera system is configured to be repositioned by pushing the camera downward along the inner sidewall of the patient bed, effectively moving it out of the caregiver's working space without requiring disassembly. In conjunction with this mechanical adjustment, the camera is connected via a magnetic quick-release connector, such as a MagSafe-type interface, which enables the data cable to detach safely from the camera when a pulling force is applied. This configuration ensures that neither the camera nor the cable sustains damage during disconnection and eliminates potential delays in patient access caused by the camera system. The combined design of the camera mount and quick-release connector enables healthcare personnel to perform time-sensitive interventions without obstruction, enhancing both patient safety and clinical efficiency.

According to one embodiment, the present invention may provide a system for continuous patient authentication to ensure that only data corresponding to the body of the patient under care is selected, captured, and logged. This continuous authentication process is executed by algorithms operating on image data acquired from one or more cameras positioned within the clinical environment. The authentication algorithms may be embedded within the camera housing itself or may run on an external computing system, such as the Machine Vision Computer or the Room Integrator Computer, which receives the camera data. In some embodiments, the system employs facial identification techniques utilizing algorithms such as Eigenfaces, Local Binary Patterns Histograms, Fisherfaces, or neural network-based models. In some implementations, data from a dedicated face camera is integrated to enhance identification accuracy. The system may alternatively or additionally apply a body-identification algorithm that leverages the patient's body shape, extracted from 2-D, 3-D, or depth camera inputs and analyzed using statistical or machine learning techniques, including differentiable rendering or shape estimation algorithms. In some embodiments, the authentication process may incorporate alternative camera inputs such as depth, spectral data, or combined ultraviolet, visible, and infrared imaging to ensure robust patient identification. The system generates a unique patient identifier, which is linked to the corresponding patient record. During each body capture event, the system performs an identity validation check against the stored identifier; if the authentication is successful, the data is logged into the patient's medical record. If authentication fails, the data is excluded from the log, thereby safeguarding against unauthorized or inaccurate recording. Additionally, the system provides visual feedback to clinical staff by illuminating LED indicators on the camera mount upon successful authentication and turning them off in the event of authentication failure. In further embodiments, data from both face and body cameras may be jointly processed using feature extraction and matching techniques or fused via early, late, or hybrid fusion methods to achieve comprehensive and tamper-resistant patient authentication. Alternatively, a statistical learning model, such as a neural network, may be trained using combined input from both the face and body cameras to create a unified authentication mechanism. Synchronization between the cameras may be achieved via fixed clocks, hardware synchronization, software synchronization, or a combination thereof, ensuring accurate temporal alignment of data streams for reliable identification and record-keeping.

According to one embodiment, the present invention may provide a system and method for structured data and image logging to enhance both clinical utility and data integrity in the context of facial expression analysis. The output data generated by facial analysis algorithms is recorded in a time-series database, allowing for chronological tracking and retrospective evaluation of patient facial states. Recognizing the inherent limitations and potential inaccuracies of automated facial analysis algorithms, a key technical feature of the invention is the concurrent storage of short video clips that capture only the patient's face immediately before and during the detected period of a change in facial expression. This enables human operators to review the isolated facial video and validate whether the algorithmic inference of a facial expression change is accurate or whether the system has produced an error, thus introducing a layer of human-in-the-loop verification. To safeguard privacy, the video clips are processed to mask and exclude any non-patient individuals from the visual data by setting the corresponding non-patient pixel regions to zero (black). The system is configured to initiate the video capture up to one minute before the detected change in facial expression and to continue recording throughout the period during which the new facial expression is maintained, ensuring both contextual clarity and privacy compliance.

According to one embodiment, the present invention may provide an advanced fluids capture camera system to automate the measurement and authentication of fluid quantities and qualities in a healthcare setting while ensuring data integrity and privacy protection. In an embodiment, the advanced fluids capture camera system uses machine vision cameras in combination with computer vision techniques to simultaneously capture and infer both the volume and qualitative attributes of fluids collected in containers, such as their color, opacity, and light transmission spectra. The camera employed may utilize CMOS or CCD sensors and is designed to meet optical character recognition (OCR) requirements at the fixed mounting distance, based on the lens configuration. The camera is securely housed within a rigid protective arm, which also shields the camera cables, and the system supports a wide range of communication protocols, including but not limited to USB, GigE, FireWire, Thunderbolt, CameraLink, and custom interfaces. The captured image data is processed by a dedicated Camera Image Processor, which may be implemented as an embedded system, FPGA, single-board computer (such as Nvidia Nano or Raspberry Pi), or as part of a common personal computer. The Camera Image Processor may also integrate hardware and software components, including statistical learning machines or neural networks, for tasks such as fluid volume inference, OCR of volume markings, and analysis of fluid quality. The Camera Image Processor or a subsequent control unit may also manage LED-based status indicators, which provide immediate visual feedback regarding the camera's power status, authentication of the fluid container, successful data capture, and error notifications. The advanced fluids capture camera system further includes a multi-spectral LED illuminator that enables the collection of rich spectral data from fluid samples. The LED array includes both narrow-band and broad-band emitters spanning ultraviolet, visible, and infrared spectra, with configurable wavelengths ranging, for example, from 390 nm to 1200 nm. The LEDs are arranged within a dedicated housing positioned within the camera's field of view and are powered through various embodiments, including DC adapters, batteries, or solar panel and battery pack combinations. The LED system is controlled by synchronization pulses generated by a microcontroller, the camera, or both, to ensure that images captured under different illumination conditions are properly aligned for multi-spectral or hyperspectral analysis. Authentication of both the camera system and the fluid containers is one of the key technical features of this invention. Each camera and its associated image processor are registered with a Room Integrator Computer (RIC) through a unique hardware identifier, ensuring that only authorized devices are permitted to upload data. Additionally, each fluid container is marked with a tamper-proof visual code such as security labels, void stickers, destructible vinyl, tamper-evidence polyester, or physical etchings which are read and verified by the camera image processor before any data logging. This dual authentication process significantly mitigates the risk of data manipulation and ensures that the fluid measurement data is reliably linked to a specific patient and container. During operation, the camera image processor conducts image pre-processing tasks, such as grayscale conversion, noise reduction, binarization, contrast adjustment, and text detection, to enhance image quality and facilitate accurate data extraction. Fluid volume is determined either by reading the calibrated markings on the container using OCR algorithms (such as Tesseract or EasyOCR) or via machine learning models trained to infer fluid levels from the image data directly, with both methods optionally combined to minimize measurement errors. Fluid quality is analyzed using multi-spectral images acquired under controlled LED illumination conditions, with each image tagged or indexed according to the wavelength or wavelength combination used during capture. This allows precise evaluation of fluid properties including color, transmission and reflection spectra, density, and opacity. All extracted data, including both quantitative measurements and multi-spectral image sets, are stored in a time-series database on the Room Integrator Computer for further clinical analysis and auditing. Depending on system configuration, the camera image processor may either reside within the RIC or operate as a separate, network-connected system. When operating as a separate unit, secure communication is ensured via encrypted wired connections (e.g., TCP/IP or UDP over Ethernet) or encrypted wireless protocols, including WiFi, Bluetooth, or other wireless communication technologies.

According to one embodiment, the present invention may provide a room monitoring system for the observation and analysis of non-patient behavior within a clinical or caregiving environment. The hardware components of the room monitoring system are substantially similar to, or in some embodiments identical to, the components used for the patient body capture system. In certain embodiments, additional cameras are strategically installed within the room and are synchronized with the patient body capture system to allow integrated or fused data analysis, enabling a holistic interpretation of the room environment and the interactions occurring therein. During operation, the room monitoring system utilizes the same patient authentication and non-patient exclusion mechanisms described in connection with the patient body capture system to ensure that only authorized individuals, such as medical staff or registered caregivers, are recorded and analyzed. In this context, non-patient staff members are authenticated using methods equivalent to those employed for patient identification, allowing the system to precisely associate the spatial regions of the captured image including the body, head, and extremities with the verified identity of the staff member. This authentication and identification process ensures that only known and approved individuals, such as the patient and registered healthcare staff, are recorded, while unregistered persons including visitors, cleaning personnel, or unauthorized individuals are excluded from the data capture. To further ensure privacy, the system applies masking techniques, such as pixelation, blurring, distortion, or other similar obfuscation methods, to selectively exclude the image segments (i.e., the pixels) corresponding to any unauthorized or unidentified individuals. In one embodiment, rather than simply masking these segments, the system replaces the obscured areas with an AI-generated video approximation that conceals the identity of non-patient and non-staff individuals while preserving the contextual relevance of their movements or presence. This approach enables behavioral analysis relating to staff performance and patient response without compromising the privacy of unrelated individuals who may enter the monitored space.

According to one embodiment, the present invention may provide a system for data capture by direct-wired connection, wherein a room integrator computer serves as the central hub for collecting, organizing, and managing data streams from various devices and computer vision systems installed within the monitored environment. The room integrator computer is configured with interface systems that enable direct wired connections to external devices, ensuring stable and secure data transfer. A critical function of the room integrator computer is to host software capable of registering the unique device ID of each connected hardware component, thereby maintaining system integrity by continuously verifying that incoming data originates from authorized, pre-registered devices. If any device is detected to have changed without a corresponding authorized update in the registry, the system is designed to trigger an alert or fail-safe mechanism. A time-series database is coupled with a data collector framework to systematically log both the data captured from wired devices and the image-based data derived from camera systems, as previously described. In various embodiments, the time-series database may include but is not limited to, commercially available solutions such as TimescaleDB, GridDB, InfluxDB, OpenTSDB, CrateDB, or any other functionally similar or custom-designed database architecture suited for high-frequency time-stamped data. To further optimize performance and minimize write-load on the database, the system may incorporate an intermediary software layer, such as Apache Kafka, Apache NiFi, Apache Storm, Apache Flink, Apache Beam, or a custom-designed equivalent, which buffers and organizes incoming data before committing it to the database. Additionally, in one embodiment, the Room Integrator Computer may be configured to synchronize its stored data with a remote data server, such as a cloud-based storage platform, ensuring secure off-site backup and enabling remote access. In alternative embodiments, the Room Integrator Computer may facilitate direct remote access for data retrieval without cloud synchronization or may combine both cloud and direct access models. In some embodiments, the room integrator computer is designed to operate in a closed network environment, where it is isolated from external networks and interfaces solely with a patient console positioned outside the monitored room.

According to one embodiment, the present invention may provide a patient console for the visualization, analysis, and reporting of patient data collected by the room integrator computer. The room integrator computer captures and integrates patient data into a unified database, such as a time-series database, and in certain embodiments, this data is visualized directly on a display connected to the room integrator computer. In some embodiments, the patient console, positioned outside the monitored room, is used to visualize the data independently. In yet another embodiment, both the room integrator computer and the patient console provide visualization functionality for the same dataset. The patient console comprises both hardware and software components configured to receive, visualize, analyze, and report patient data. In various embodiments, the patient console may be implemented as a tablet computer, including but not limited to devices such as the Apple iPad, and iPad Pro, Android-based tablets like the Samsung Galaxy Tablet, or tablets running Microsoft Windows, Linux, or Unix-based operating systems such as BSD. In alternative embodiments, the Patient Console may be a standalone personal computer, such as an IBM-compatible or Macintosh computer, also capable of running Windows, Linux, or Unix operating systems. Hardware configurations are adaptable to meet the performance and display requirements of the associated software. In some embodiments, the patient data is displayed on high-resolution digital displays, which may include touch or pen-input functionality for user interaction. Additionally, the system may incorporate various input devices, such as a mouse, trackball, trackpad, foot-based controllers, or advanced gesture recognition systems including RF-based technologies like GestIC or video-based systems employing 3D depth cameras, such as Intel's RealSense, combined with gesture recognition software. The patient console has access to all data captured and stored by the room integrator computer and, in certain embodiments, also retrieves supplementary patient data from Electronic Medical Records (EM R) systems, such as EPIC. Data obtained from EM R systems are synchronized and timestamped to ensure temporal consistency with the locally stored data. The patient console also allows manual data entry by authorized medical personnel. Such entries are authenticated via methods such as ID card scanning, user credentials, facial recognition, or similar verification systems, and are stored along with the identity of the person entering the data, ensuring accountability and data integrity. Multiple patient data sources are integrated such as physiological sensor data, video streams of patient faces and body movements, and other patient-monitoring devices to infer the patient's mental and emotional state. The system employs algorithms to correlate facial expressions, body movements, and vital sign statistics such as heart rate, heart rate variability, blood pressure, blood oxygenation, EEG, and ECG to assess and classify patient states. In one embodiment, this inference is performed using discrete systems programmed to recognize value ranges or apply dynamic programming. In other embodiments, statistical learning models, including but not limited to neural networks, general linear models, and both linear and nonlinear statistical frameworks whether frequentist, Bayesian, or hybrid approaches are employed to compute patient state predictions. These predictions include labels for mental and emotional states, such as comfort, distress, pain, sleep stages, delirium, depressive states, or other clinically relevant conditions, along with associated confidence metrics or error probabilities. The system also enables clinicians to review the evidence used in state determination and allows authorized users to re-weight or exclude specific data sources to refine these inferences. The resulting state assessments are then stored by the room integrator computer as part of the patient's record. The patient console also includes advanced data visualization capabilities to enable authorized users to interactively review all patient data, including time-series datasets and event-driven records such as medical imaging results. In some embodiments, the present system may enable the visualization of multiple data streams in a format familiar to domains such as financial trading but not previously applied to medical patient monitoring. Users can select multiple variables for simultaneous display, adjust scales for comparative analysis, and zoom across various time windows to focus on specific periods or data ranges. Furthermore, users can jointly visualize datasets from recurring events, such as periods following drug administration, to infer cause-and-effect relationships between interventions and patient responses. Beyond visualization, the patient console incorporates a comprehensive suite of statistical and inferential analysis tools to allow clinical staff to perform advanced data interrogation without requiring external software. These tools include but are not limited to, segmented regression, simple and multiple regression, multivariate regression, principal components analysis, independent components analysis, cluster analysis, t-SNE, manifold learning, topological data analysis, and other higher-order statistical techniques. Users can also apply data preprocessing methods such as smoothing, filtering, and feature extraction using algorithms ranging from traditional splines and Fourier filtering to modern machine learning models, including neural networks. Another key technical feature is the system's support for modular analysis pipelines, which can be loaded, executed, saved, and shared. These pipelines may be obtained from third parties or shared via an integrated application marketplace (“app store”), enabling clinicians to adopt, distribute, and apply standardized data processing and analysis workflows directly within the Patient Console.

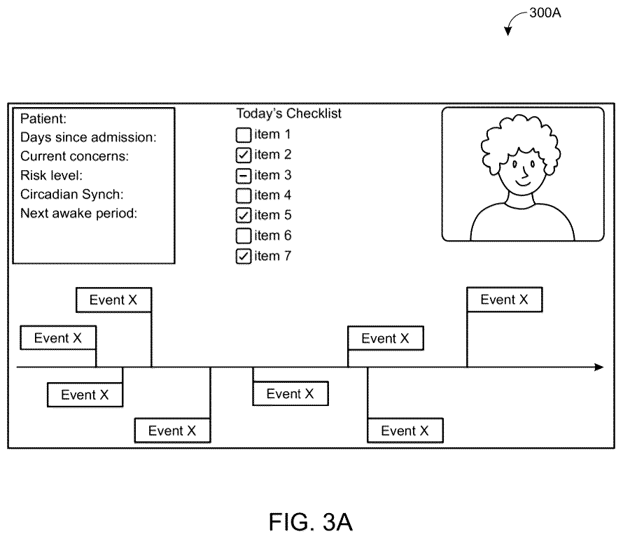

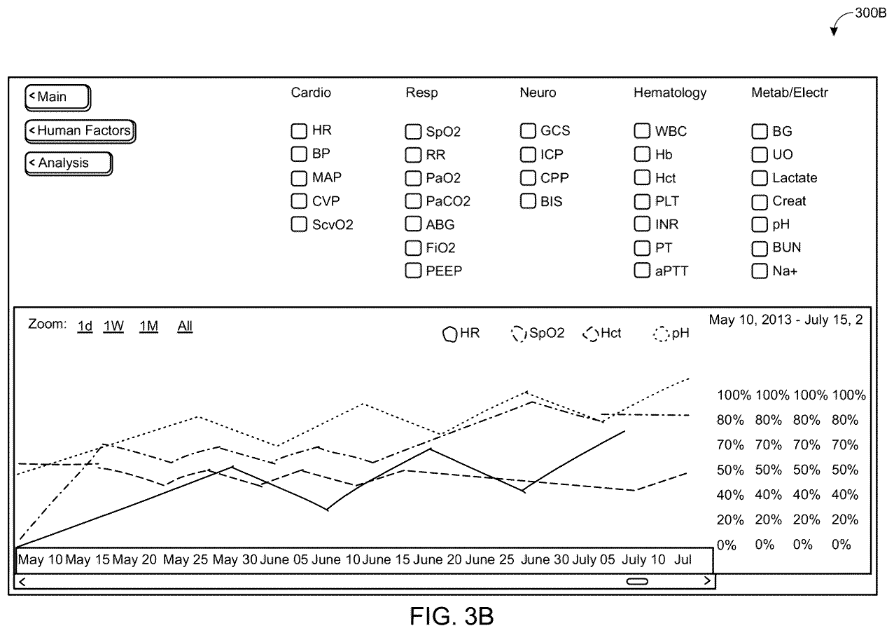

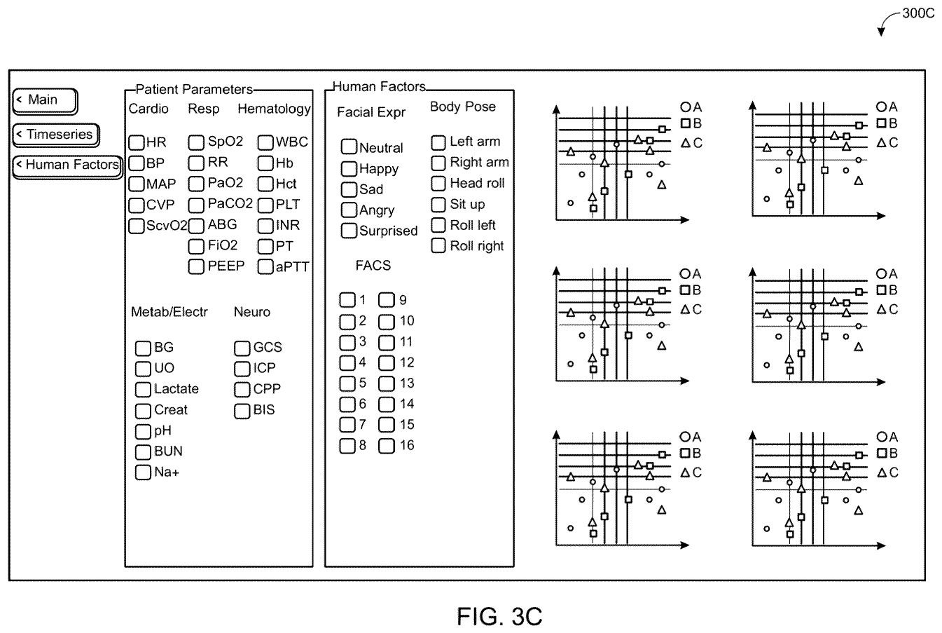

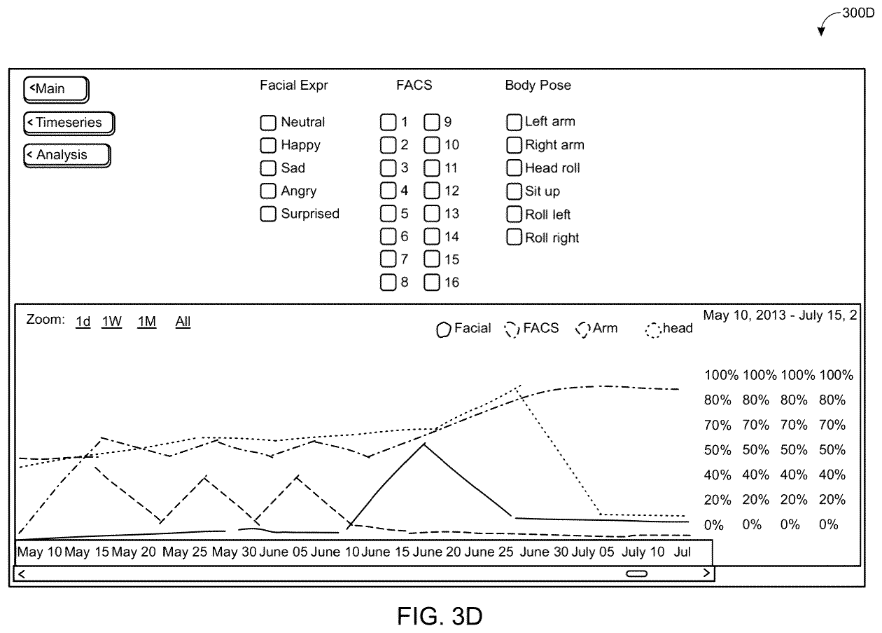

FIG. 3A is a diagram that depicts a user interface 300A of a main console pertaining to patient registration and key event overview, in accordance with an embodiment of the disclosure. The main console provides a centralized dashboard comprising (i) patient registration details, (ii) an event timeline, (iii) a structured daily checklist, and (iv) live patient imagery. As shown, the patient registration section captures essential contextual parameters such as days since admission, current clinical concerns, patient-specific risk level, circadian synchronization status, and estimated next awake period. This metadata supports intelligent scheduling, monitoring, and alert generation by the system. A chronological event timeline is depicted along the lower half of the interface, showing system-detected or manually logged clinical events (“Event X”) over time. This visual format allows clinicians to understand temporal correlations and identify patterns in patient care activities or system-detected anomalies. The main console supports real-time clinical decision-making and provides auditability of patient interactions, and enables seamless synchronization between automated data capture and human-in-the-loop workflows.

FIG. 3B is a diagram that depicts a user interface 300B of clinical parameters and interventions, in accordance with an embodiment of the disclosure. The interface enables real-time and retrospective monitoring of various physiological metrics across multiple clinical domains, including cardiovascular, respiratory, neurological, hematological, and metabolic/electrolyte categories. The interface presents a modular checklist of selectable parameters such as heart rate (HR), blood pressure (BP), oxygen saturation (SpO2), and hematocrit (Hct) to allow dynamic filtering and customized data display based on clinical relevance. This interface further provides a time-series graph plotting selected parameters and also provides a visual trend analysis of interventions (e.g., medication dosages or fluid levels) in relation to patient responses over a defined period. In some embodiments, the system may compute derived metrics, perform alerts on outlier patterns, and align interventions with underlying physiological changes.

FIG. 3C is a diagram that depicts a user interface 300C of an example of an analysis window, looking at the correlation between parameters, in accordance with an embodiment of the disclosure. The interface includes a comprehensive selection panel that allows clinicians or systems to enable/disable specific physiological parameters (e.g., HR, SpO₂, ICP, Na⁺), body metrics (e.g., pose), and emotional or facial expression indicators (e.g., neutral, happy, sad, FACS codes). Further, this interface visually depicts multiple scatter or correlation plots (graphical windows) showing the relationships between selected variables identified by colored data points plotted across axes corresponding to parameters A, B, and C. These visualization windows may support time-aligned or event-triggered comparisons.

FIG. 3D is a diagram that depicts a user interface 300D of patient state parameters, in accordance with an embodiment of the disclosure. This is a graphical user interface for monitoring and analyzing patient state parameters over time, with a focus on visual behavior, facial expressions, and body pose metrics. The graph presents selectable indicators, including facial expressions (e.g., neutral, happy, sad), Facial Action Coding System (FACS) units (e.g., AU01-AU16), and body pose classifications (e.g., left/right arm raised, head roll, sit-up posture), which may be derived through computer vision or sensor fusion techniques. Further, this graph also displays a time-series visualization of selected patient state parameters across a configurable time window (e.g., daily, weekly, or monthly views). The curves represent the temporal evolution of state detection percentages, such as frequency or confidence levels for “Facial,” “FACS,” “Arm,” and “Head” positions.

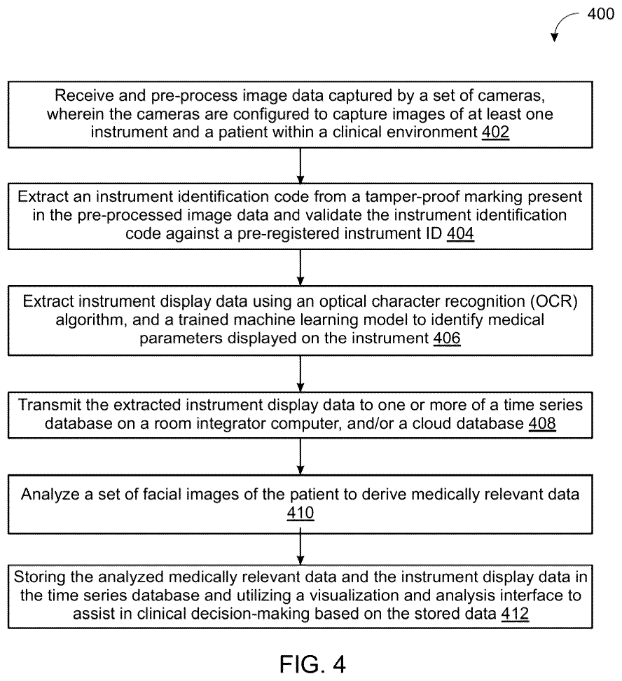

FIG. 4 illustrates a flowchart 400 that illustrates a method for monitoring a patient, in accordance with an embodiment of the disclosure. FIG. 4 is explained in conjunction with elements from FIG. 1, FIG. 2, and FIG. 3. At 402, the computer-implemented method includes receiving and pre-processing, by a computer, image data captured by a set of cameras. The cameras are configured to capture images of at least one instrument and a patient within a clinical environment. At 404, the computer-implemented method further includes extracting, by the computer, an instrument identification code from a tamper-proof marking present in the pre-processed image data and validating the instrument identification code against a pre-registered instrument ID. In one embodiment, the system is configured to continuously authenticate medical instruments that appear in the camera feed. The extracted identifier is validated in real-time against a database of pre-registered instrument identification codes. This continuous verification mechanism ensures that only authorized and verified instruments are recognized and logged by the system, thereby enhancing traceability, safety, and regulatory compliance in the clinical environment. In another embodiment, the method also performs continuous authentication of the patient whose facial and/or bodily data is being captured. The captured identity information is matched against a pre-registered patient profile to ensure the correct association of recorded data with the intended individual. This continuous patient verification process helps maintain the integrity of clinical records and prevents misidentification errors that could otherwise compromise patient care. Furthermore, the method selectively excludes non-patient entities from being captured or processed in the visual data stream. By limiting data capture to only the identified patient, the present invention preserves the confidentiality of non-consenting individuals in the monitored environment.

At 406, the computer-implemented method further includes extracting, by the computer, instrument display data using one or more of an optical character recognition (OCR) algorithm, and a trained machine learning model to identify a set of medical parameters displayed on the instrument. Examples of the medical parameters include, but are not limited to, the patient's facial state, body posture, limb positioning, and overall pose. Additionally, patient vitals are captured either directly—through wired or networked connections—or indirectly via camera-based monitoring. At 408, the computer-implemented method further includes transmitting, by the computer, the extracted instrument display data to a time series database on a room integrator computer. At 410, the computer-implemented method further includes analyzing, by the computer, a set of facial images of the patient to derive medically relevant data. At 412, the analyzed medically relevant data and the instrument display data are stored in the time series database and a visualization and analysis interface is utilized to assist in clinical decision-making based on the stored data.

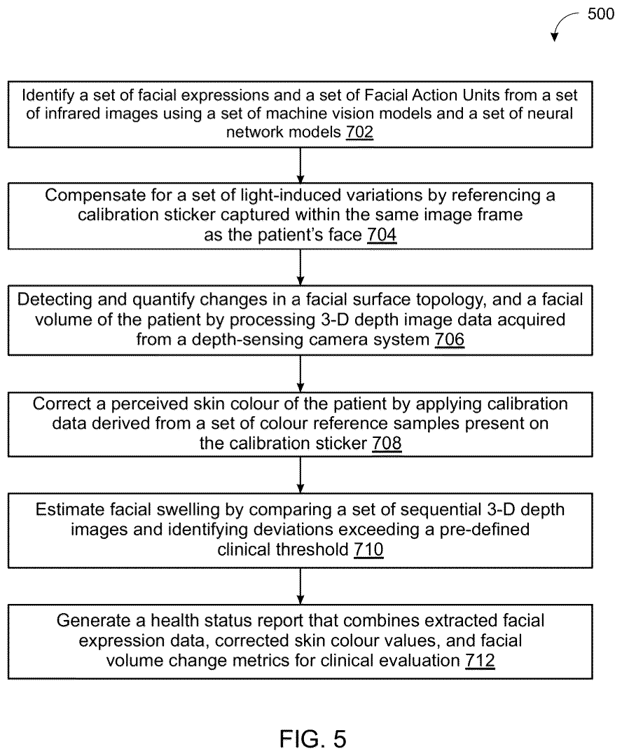

FIG. 5 illustrates a flowchart 500 that illustrates an exemplary method for analyzing the facial images of the patient, in accordance with an embodiment of the disclosure. FIG. 5 is explained in conjunction with elements from FIG. 1, FIG. 2, FIG. 3, and FIG. 4. At 502, changes in a facial surface topology, and a facial volume of the patient are detected and quantified by processing 3-D depth image data acquired from a depth-sensing camera system. At 504, a perceived skin colour of the patient is corrected by applying calibration data derived from a set of colour reference samples present on the calibration sticker. At 506, facial swelling is estimated by comparing a set of sequential 3-D depth images and identifying deviations exceeding a pre-defined clinical threshold. At 508, a health status report is generated that combines extracted facial expression data, corrected skin colour values, and facial volume change metrics for clinical evaluation. The extracted instrument display data is transmitted from the computer to the room integrator computer via one or more of: an encrypted cabled connection, and an encrypted wireless connection. The infrared images are captured by a compound camera system comprising a set of infrared cameras, a set of 3D cameras, and a set of multispectral imaging systems. The calibration sticker includes an array of colour samples with predetermined and fixed reflectance properties.

In one embodiment, the visual data is indicative of the patient's physiological or behavioral state is captured via one or more imaging devices and is combined with physiological data obtained from vitals monitoring equipment such as heart rate, respiratory rate, blood pressure, oxygen saturation, and temperature monitors and/or with treatment-related data, including but not limited to medication types, dosages, administration frequencies, and therapeutic interventions. This multimodal data is subjected to data fusion techniques, wherein temporally and contextually aligned information from these distinct sources is aggregated and processed to infer the real-time state of the patient. In one implementation, this fusion-based analysis may employ rule-based logic, statistical correlation models, or machine learning algorithms to derive clinically relevant insights regarding the patient's current condition, trajectory of recovery or deterioration, and responsiveness to administered treatments. In an embodiment, the facial and body capture information utilizes integration (fusion) across available data sources. For example, patient vitals may be integrated with facial and body data to enable more accurate inference of possible expressions of pain or other physiological states.

In an embodiment, the extracted instrument display data is transmitted from the computer to the room integrator computer via one or more of: an encrypted cabled connection, and an encrypted wireless connection. In an embodiment, the infrared images are captured by a compound camera system comprising a set of infrared cameras, and a set of multispectral imaging systems. In an embodiment, the calibration sticker comprises an array of colour samples with predetermined and fixed reflectance properties.

According to one or more embodiments of the disclosure, a computer program product for monitoring a patient is described. The computer program product includes a computer-readable storage media having program instructions stored on the computer-readable storage media to perform operations. The operations include receive and pre-process image data captured by a set of cameras. The cameras are configured to capture images of at least one instrument and a patient within a clinical environment. The operations further include extract an instrument identification code from a tamper-proof marking present in the pre-processed image data and validate the instrument identification code against a pre-registered instrument ID. The operations further include extract instrument display data using one or more of an optical character recognition (OCR) algorithm, and a trained machine learning model to identify a set of medical parameters displayed on the instrument. The operations further include transmit the extracted instrument display data to a time series database on a room integrator computer. The operations further include analyze a set of facial images of the patient to derive medically relevant data. The operations further include storing the analyzed medically relevant data and the instrument display data in the time series database and utilizing a visualization and analysis interface to assist in clinical decision-making based on the stored data.

The descriptions of the various embodiments of the disclosure have been presented for purposes of illustration but are not intended to be exhaustive or limited to the embodiments disclosed. M any modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A computer system, comprising:
a memory to store machine-readable instructions pertaining to monitoring of a patient; and
a processor set configured to:
receive and pre-process image data captured by a set of cameras, wherein the set of cameras are configured to capture images of at least one instrument and a patient within a clinical environment;
extract an instrument identification code from a tamper-proof marking present in the pre-processed image data and validate the instrument identification code against a pre-registered instrument ID;
extract instrument display data using one or more of an optical character recognition (OCR) algorithm, and a trained machine learning model to identify a set of medical parameters displayed on the instrument;
transmit the extracted instrument display data to a time series database on at least one of a room integrator computer, and a cloud database;
analyze a set of facial images of the patient to derive medically relevant data; and
store the analyzed medically relevant data and the instrument display data in the time series database and utilize a visualization and analysis interface to assist in clinical decision-making based on the stored data.

2. The computer system of claim 1, wherein the processor set is configured to:
identify a set of facial expressions and a set of Facial Action Units from a set of infrared images using one or more of a set of machine vision models and a set of neural network models; and
compensate for a set of light-induced variations by referencing a calibration sticker captured within the same image frame as the patient's face.

3. The computer system of claim 1, wherein the processor set is configured to:
detect and quantify changes in a facial surface topology, and a facial volume of the patient by processing 3-D depth image data acquired from a depth-sensing camera system; and
correct a perceived skin colour of the patient by applying calibration data derived from a set of colour reference samples present on the calibration sticker.

4. The computer system of claim 1, wherein the processor set is configured to:
estimate facial swelling by comparing a set of sequential 3-D depth images and identify deviations exceeding a pre-defined clinical threshold; and
generate a health status report that combines extracted facial expression data, corrected skin colour values, and facial volume change metrics for clinical evaluation.

5. The computer system of claim 1, wherein the extracted instrument display data is transmitted from the processor set to the room integrator computer via one or more of: an encrypted cabled connection, and an encrypted wireless connection.

6. The computer system of claim 2, wherein the infrared images are captured by a compound camera system comprising a set of infrared cameras, a set of 3D cameras, and a set of multispectral imaging systems.

7. The computer system of claim 2, wherein the calibration sticker comprises an array of colour samples with predetermined and fixed reflectance properties.

8. A computer-implemented method, the method comprising:
receiving and pre-processing, by a computer, image data captured by a set of cameras, wherein the set of cameras are configured to capture images of at least one instrument and a patient within a clinical environment;
extracting, by the computer, an instrument identification code from a tamper-proof marking present in the pre-processed image data and validating the instrument identification code against a pre-registered instrument ID;
extracting, by the computer, instrument display data using one or more of an optical character recognition (OCR) algorithm, and a trained machine learning model to identify a set of medical parameters displayed on the instrument;
transmitting, by the computer, the extracted instrument display data to a time series database on at least one of a room integrator computer, and a cloud database;
analyzing, by the computer, a set of facial images of the patient to derive medically relevant data; and
storing the analyzed medically relevant data and the instrument display data in the time series database and utilizing a visualization and analysis interface to assist in clinical decision-making based on the stored data.

9. The computer-implemented method of claim 8, further comprising:
identifying, by the computer, a set of facial expressions and a set of facial action units from a set of infrared images using one or more of a set of machine vision models and a set of neural network models; and
compensating, by the computer, for a set of light-induced variations by referencing a calibration sticker captured within the same image frame as the patient's face.

10. The computer-implemented method of claim 8, further comprising:
detecting and quantifying, by the computer, changes in a facial surface topology, and a facial volume of the patient by processing 3-D depth image data acquired from a depth-sensing camera system; and
correcting, by the computer, a perceived skin colour of the patient by applying calibration data derived from a set of colour reference samples present on the calibration sticker.

11. The computer-implemented method of claim 8, further comprising:
estimating, by the computer, facial swelling by comparing a set of sequential 3-D depth images and identifying deviations exceeding a pre-defined clinical threshold; and
generating, by the computer, a health status report that combines extracted facial expression data, corrected skin colour values, and facial volume change metrics for clinical evaluation.

12. The computer-implemented method of claim 8, wherein the extracted instrument display data is transmitted from the computer to the room integrator computer via one or more of: an encrypted cabled connection, and an encrypted wireless connection.

13. The computer-implemented method of claim 9, wherein the infrared images are captured by a compound camera system comprising a set of infrared cameras, a set of 3D cameras, and a set of multispectral imaging systems.

14. The computer-implemented method of claim 9, wherein the calibration sticker comprises an array of colour samples with predetermined and fixed reflectance properties.

15. A computer program product comprising at least one non-transitory computer-readable storage medium having computer-executable program code instructions stored therein, the computer-executable program code instructions comprising program code instructions to:
receive and pre-process image data captured by a set of cameras, wherein the set of cameras are configured to capture images of at least one instrument and a patient within a clinical environment;
extract an instrument identification code from a tamper-proof marking present in the pre-processed image data and validate the instrument identification code against a pre-registered instrument ID;
extract instrument display data using one or more of an optical character recognition (OCR) algorithm, and a trained machine learning model to identify a set of medical parameters displayed on the instrument;
transmit the extracted instrument display data to a time series database on at least one of a room integrator computer, and a cloud database;
analyze a set of facial images of the patient to derive medically relevant data; and
store the analyzed medically relevant data and the instrument display data in the time series database and utilize a visualization and analysis interface to assist in clinical decision-making based on the stored data.
